(19) United States
(12) Patent Application Publication
(10) Pub. No.: US 2020/0237613 A1
(43) Pub. Date: Jul. 30, 2020

(54) SKIN TREATMENT DEVICES AND METHODS OF USE

(71) Applicant: Filip Sedic, Stockholm (SE)

(72) Inventor: Filip Sedic, Stockholm (SE)

(21) Appl. No.: 16/577,042

(22) Filed: Sep. 20, 2019

Related U.S. Application Data
(60) Provisional application No. 62/796,943, filed on Jan. 25, 2019.

(51) Int. Cl. A61H 23/02; A61H 23/00; A61N 1/36

(52) U.S. Cl. CPC A61H 23/0218; A61H 23/006; A61N 1/36014; A61H 2201/0207; B08B 1/002; A61H 2201/5082; A61H 2201/169; A61H 2201/0153; A61H 2201/10

(57) ABSTRACT

An example skin treatment device includes a surface with at least one textured portion for transmitting vibrations and/or pulsations to the skin. The skin treatment device includes at least one motor for generating such vibrations and/or pulsations. The skin treatment device also includes a heating element and a heat transfer mechanism to produce and transfer heat therapy to a user. The skin treatment device further includes at least one stimulation mechanism configured to transfer electrical current stimulation to a user.

### CROSS-REFERENCE TO RELATED APPLICATIONS

This application claims the benefit of U.S. Provisional Patent Application No. 62/796,943, filed Jan. 25, 2019, which is incorporated into this disclosure in its entirety.

### FIELD

The disclosure relates generally to the field of skin treatment devices and methods of use. More particularly, the disclosure relates to devices suitable for use on the human face and body and various methods of using the same.

### BACKGROUND

Skin health and appearance is an important aspect of many beauty regimens. As a person ages, his or her skin tends to develop wrinkles, sag, become rough, and/or develop various spots or marks. Stimulating skin, facial, tissue, and/or muscle cells through various means such as through massage therapy, electrical current stimulation, and/or heat therapy can produce positive effects on the skin. Such positive effects, for example, may include one or more of: increased collagen and/or elastin production, reduction of skin inflammation, improved muscle tone, improved blood flow, a youthful appearance, elimination of fine lines and wrinkles, and tightening of the skin.

Skin cleanliness is also an important aspect of many beauty regimes. Skin cleansing routines typically involve various creams and or lotions which are applied to the skin by hand, brush, or sponge. Application of such creams or lotions is often ineffective at removing grease, oils, and other contaminants. Electric devices tend to be more effective at cleansing the skin than manual techniques.

Several devices exist that may massage, provide heat therapy to, provide electrical current stimulation to, or clean the skin. Known devices do not, however, include each of these functions. A need exists, therefore, for improved skin treatment devices that provide massage, heat therapy, electrical current stimulation, and cleanliness functionalities.

### BRIEF SUMMARY OF SELECTED EXAMPLES

Various example skin treatment devices and methods of use are described.

An example skin treatment device having a main body configured to treat the skin of a user comprises a substantially flat base configured to stand unaided on a substantially flat surface, said main body having a cross-sectional shape that is longer in a first direction substantially parallel to the base than in a second direction substantially parallel to the base, a silicone exterior defining a first set of touch-points integrally formed with the silicone exterior, the first set of touch-points being disposed on a first side of said main body, an oscillating motor disposed within said main body, the oscillating motor configured to produce pulsations of said skin treatment device, a heating element disposed adjacent the first side of the main body, the heating element configured to produce heat, and a heat transfer mechanism disposed adjacent the heating element and silicone exterior, the heat transfer mechanism configured to transfer the heat to said skin.

Another example skin treatment device having a main body configured to treat the skin of a user comprises a substantially flat base configured to stand unaided on a substantially flat surface, said main body having a cross-sectional shape that is longer in a first direction substantially parallel to the base than in a second direction substantially parallel to the base, a silicone exterior defining a first set of touch-points integrally formed with the silicone exterior, the first set of touch-points being disposed on a first side of said main body, an oscillating motor disposed within said main body, the oscillating motor configured to produce pulsations of said skin treatment device, and a stimulation mechanism disposed on a second side of said main body, the stimulation mechanism configured to transfer electrical current stimulation to said skin.

Another example skin treatment device having a main body configured to treat the skin of a user comprises a substantially flat base configured to stand unaided on a substantially flat surface, said main body having a cross-sectional shape that is longer in a first direction substantially parallel to the base than in a second direction substantially parallel to the base, a silicone exterior having a first side defining a first set of touch-points integrally formed with the silicone exterior, the first set of touch-points being disposed on a first side of said main body, an oscillating motor disposed within said main body, the oscillating motor configured to produce pulsations of the skin treatment device, a heating element disposed adjacent the first side of the main body, the heating element configured to produce heat, a heat transfer mechanism disposed adjacent the heating element and silicone exterior, the heat transfer mechanism configured to transfer the heat to said skin, a stimulation mechanism disposed on a second side of the silicone exterior, the stimulation mechanism configured to transfer electrical current stimulation to said skin, and a second stimulation mechanism disposed on a second side of the silicone exterior, the second stimulation mechanism configured to transfer electrical current stimulation to said skin.

Additional understanding of claimed devices and methods may be obtained by reviewing the detailed description of selected examples, below, with reference to the appended drawings.

### DESCRIPTION OF FIGURES

FIG. 1 is a perspective view of a first example skin treatment device.

FIG. 2 is an end view of the skin treatment device illustrated in FIG. 1.

FIG. 3 is another end view of the skin treatment device illustrated in FIG. 1.

FIG. 4 is a side view of the skin treatment device illustrated in FIG. 1.

FIG. 5 is another side view of the skin treatment device illustrated in FIG. 1.

FIG. 6 is a top view of the skin treatment device illustrated in FIG. 1.

FIG. 7 is a bottom view of the skin treatment device illustrated in FIG. 1.

FIG. 8 is a sectional view of the skin treatment device illustrated in FIG. 4, taken along line 8-8.

FIG. 9 is an exploded view of the skin treatment device illustrated in FIG. 1.

FIG. 10 is a diagram illustrating components of a networked skin treatment device in an example embodiment.

FIG. 11 is a perspective view of a second example skin treatment device.

FIG. 12 is an end view of the skin treatment device illustrated in FIG. 11.

FIG. 13 is another end view of the skin treatment device illustrated in FIG. 11.

FIG. 14 is a side view of the skin treatment device illustrated in FIG. 11.

FIG. 15 is another side view of the skin treatment device illustrated in FIG. 11.

FIG. 16 is a top view of the skin treatment device illustrated in FIG. 11.

FIG. 17 is a bottom view of the skin treatment device illustrated in FIG. 11.

FIG. 18 is a perspective view of a third example skin treatment device.

FIG. 19 is an end view of the skin treatment device illustrated in FIG. 18.

FIG. 20 is another end view of the skin treatment device illustrated in FIG. 18.

FIG. 21 is a side view of the skin treatment device illustrated in FIG. 18.

FIG. 22 is another side view of the skin treatment device illustrated in FIG. 18.

FIG. 23 is a top view of the skin treatment device illustrated in FIG. 18.

FIG. 24 is a bottom view of the skin treatment device illustrated in FIG. 18.

FIG. 25 is a flowchart representation of an example method of using a skin treatment device.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION OF SELECTED EXAMPLES

The following detailed description and the appended drawings describe and illustrate various skin treatment devices and methods of use. The description and drawings are provided to enable one skilled in the art to make and use one or more example skin treatment devices. They are not intended to limit the scope of the claims in any manner.

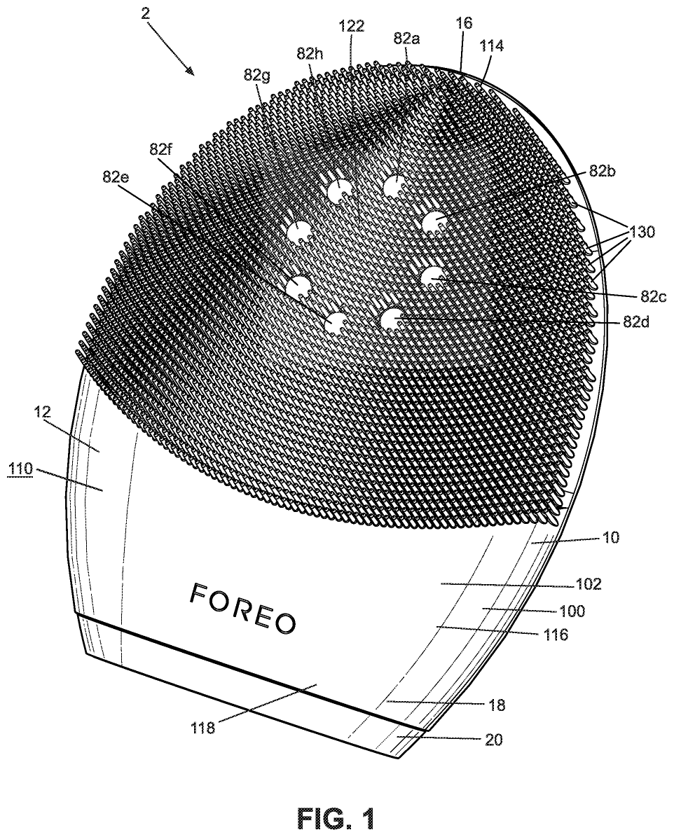

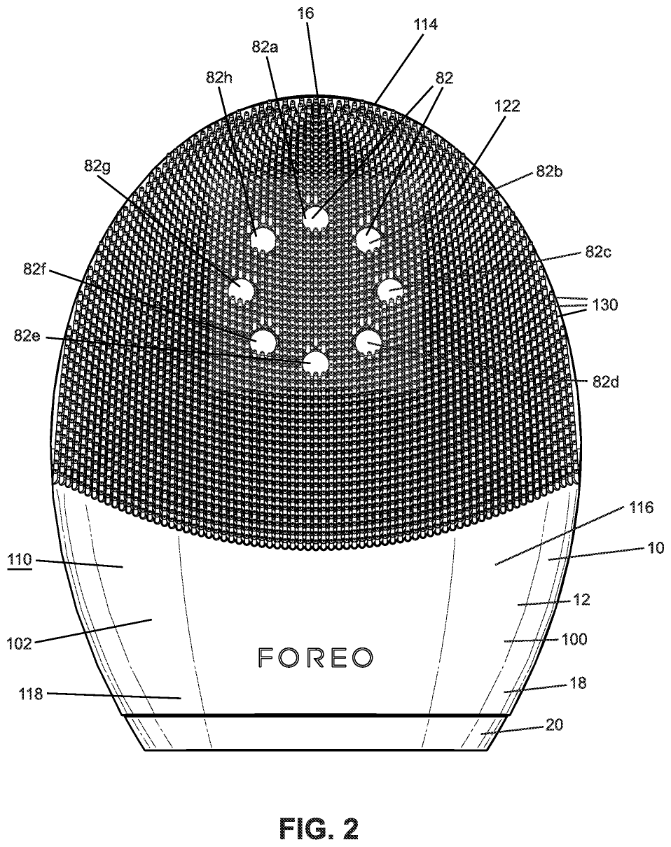

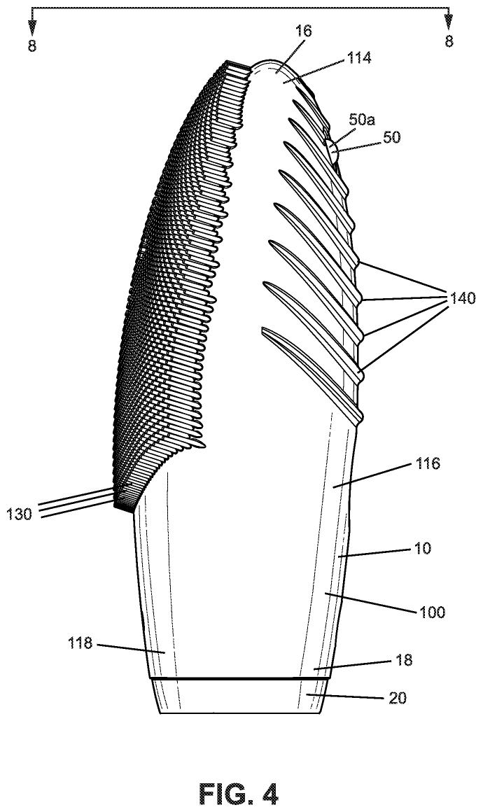

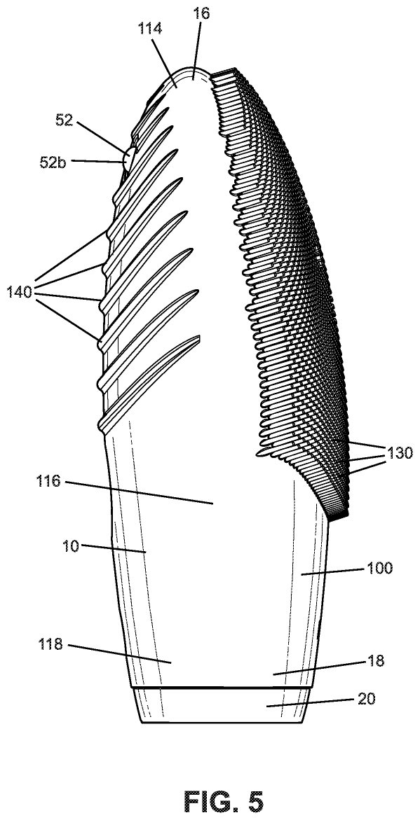

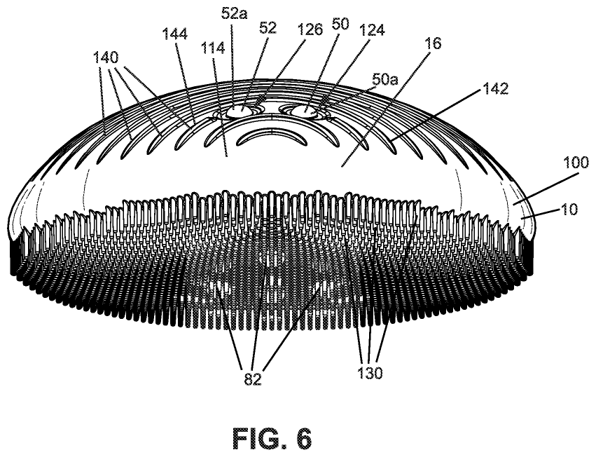

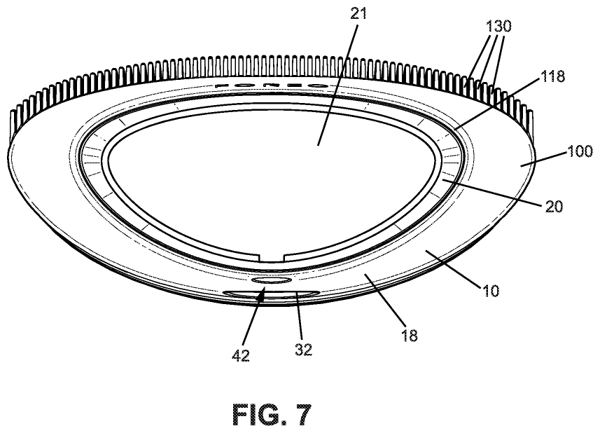

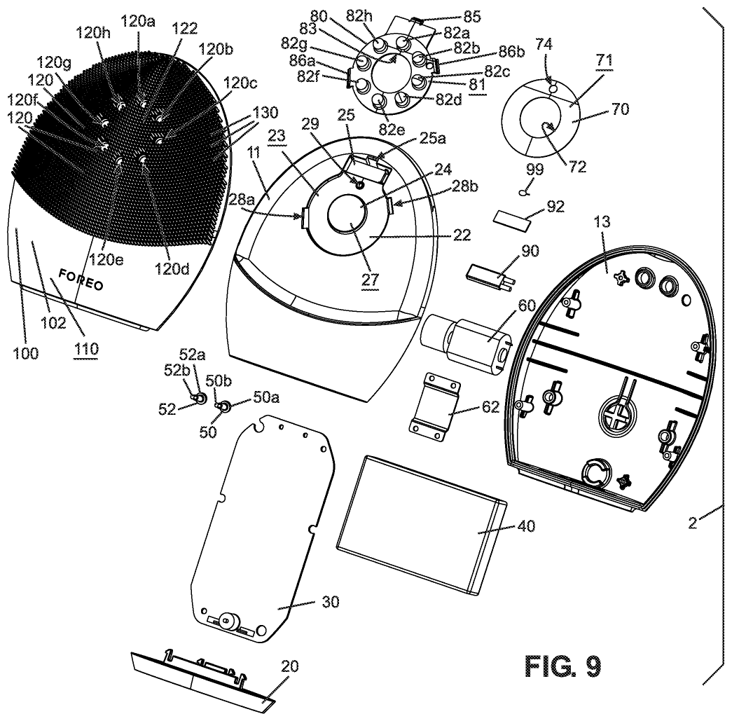

FIGS. 1, 2, 3, 4, 5, 6, 7, 8, and 9 illustrate an example skin treatment device 2 or one or more components thereof. The skin treatment device 2 (sometimes referred to as “the device 2” herein) includes a main body 10 having a first side 12, a second side 14 substantially opposite the first side 12, an upper portion 16, and a lower portion 18 that is substantially opposite the upper portion 16. The lower portion 18 includes a base 20.

The main body 10 may have any suitable shape and may be comprised of any number of pieces. In the illustrated embodiment, the main body 10 is taller than it is wide and is comprised of a first piece 1 and a second piece 13; in other embodiments, however, the main body may be wider than it is tall and may be comprised of one, three, four, or more than four pieces. The first and second pieces 11, 13 are secured to one another such that they may not be separated once the device 2 has been assembled. They are also secured to the base 20. The shape of the device 2 allows the user to hold the skin treatment device 2 in the palm of his or her hand, possibly with fingers splayed along the second side 14 of the device 2 and a thumb against a user control (described below) on the second side 14. The main body may have a substantially oval shape in some embodiments and a substantially round shape in others. The first and second pieces may be comprised of plastic or any other suitable material. A particular device may be configured to allow the user to easily hold the skin treatment device in the palm of his or her hand and reach his or her fingers around both sides of the device for easy and ergonomic manipulation of the device against the skin. The first and second pieces may be secured to one another via one or more adhesives, a snap-fit structure, and/or any mechanical attachment mechanism in various embodiments.

The illustrated skin treatment device 2 is configured to stand on a substantially flat surface, such as a table, via its base 20, which includes a substantially flat bottom 21. Such a configuration is convenient to users because it allows the device 2 to be easily stood upon a surface via the base 20 when it is not in use. This prevents the exterior covering 100 of the device 2 from contacting a potentially dirty or un-hygienic surface. The base 20 is also substantially transparent in the illustrated embodiment. In alternative embodiments, the skin treatment device may not include a base and may be configured such that it is not capable of standing on a substantially flat surface. The first and second pieces may be secured to the base via one or more adhesives, a snap-fit structure, and/or any mechanical attachment mechanism in various embodiments.

A skilled artisan will be able to select a suitable design for the device according to a particular example based on various considerations, including the strength of the motor and the particular heating element used in the device. In various embodiments, the device can be configured to have any suitable shape, including a substantially oval shape, a substantially round shape, a triangular shape, a substantially leaf-like shape, a curved shape, and so forth. In different embodiments, the device may have any suitable height (extending from the bottom of the base to the top of the upper portion) and width (extending across the widest portion of the first side), as well. Suitable skin treatment device heights include heights between about 3 centimeters (“cm”) and about 20 cm, heights between about 6 cm and about 17 cm, and heights between about 9 cm and about 14 cm. Suitable skin treatment device widths include widths between about 3 cm and about 15 cm, widths between about 6 cm and about 12 cm, and widths between about 9 cm and about 11 cm. In another embodiment, the base may be non-transparent and/or may be partially or totally covered by the exterior covering.

The first piece 11 of the main body 10 also includes an indented portion 22 configured to secure a heating element 70 on the surface 23 of the first piece 11 and a heat transfer mechanism 80 adjacent the first piece 11 and the heating element 70. The indented portion 22 is substantially circular in shape (though it does define two cutouts 28a, 28b about its circumference) and, cooperatively with the main body 10, defines a protrusion 24 in its interior. The indented portion 22 also directly adjacent and cooperatively formed with an attachment portion 25 that is configured to allow the heat transfer mechanism 80 to be secured to the first piece 11. The attachment portion 25 is substantially rectangular in shape, though it does define a cutout 25a along its perimeter. The protrusion 24 is substantially cylindrical in shape and includes a flat, circular upper surface 27, as well. The protrusion 24 extends away from the surface of the indented portion 22 to such a degree that the passageways of the heating element 70 and heat transfer mechanism 80 (described in greater detail below) may snugly fit within the indented portion 22 and about the protrusion 24, helping to secure the same on the first piece 11. Optionally, in some embodiments the indented portion may include a recess that may be aligned with a second passageway (described in greater detail below) of the heating element that will allow the heating element to be secured to the first piece via a pin or other similar mechanism. Additionally, the attachment portion 25 is configured such that an end of the heat transfer mechanism 80 may snugly fit into the attachment 25, allowing the same to be secured to the first piece 11 over the heating element 70. In some embodiments, the attachment portion attaches to the heat transfer mechanism via a snap-fit design. A skilled artisan will be able to suitably form the indented and attachment portions according to a particular example based on various considerations, including the size and shapes of the heating element and heat transfer mechanism. In various embodiments, the indented portion may have any shape, including that of an oval, ellipse, triangle, rectangle, square, pentagon, circle, and pill. In other embodiments, the attachment portion may have any shape, including that of an oval, ellipse, triangle, rectangle, square, pentagon, circle, and pill. In various embodiments, one or both of the heating element and heat transfer mechanism may be secured to one or both of the indented portion and/or attachment portion via any mechanism, including one or more mechanical attachments, pins, adhesives, and/or snap-fit structures. In other embodiments, the attachment portion and indented portion may not be cooperatively formed and may be disposed upon different portions of the first piece; additionally, in other embodiments one or both of these portions may not be included. Optionally, in some embodiments a silicone membrane and/or piece may be placed upon the surface of the indented portion (potentially including the surface and/or sides of the protrusion) to prevent the heating element from melting or otherwise negatively impacting the first piece.

The main body 10 of the skin treatment device 2 defines a cavity 15, which houses a controller 30, a power source 40, a first stimulation mechanism 50, a second stimulation mechanism 52, a motor 60, the heating element 70, and the heat transfer mechanism 80.

The motor 60 is partially disposed within in a frame 62 disposed within the cavity 15 of the main body 10. The frame 62 is configured to support the motor 60 and keep the motor 60 secured at a particular location inside the main body 10, as well as provide general structural support to one or more portions of the device 2. In some embodiments, there is more than one motor, in embodiments containing multiple motors, the motors may vary from one another in frequency output. The controller 30 (described in greater detail below), provides instructions to the motor 60, which is powered by the power source 40. A high or low-frequency motor may be used to create pulsations that vibrate the skin treatment device in various embodiments. The motor 60 in this embodiment provides vibration frequencies between about 50 hertz (“Hz”) and about 300 Hz, though pulsation frequencies higher and lower than this range may also provide different types of skin cleansing and/or massage. When the skin treatment device 2, and the exterior covering 100 in particular, is applied to the body, such as the face or neck, the pulsations provide a thorough massaging of the skin and stimulation of skin cells, tissues, muscle cells, and various other portions of a user. A skilled artisan will be able to select a suitable motor and/or motors and vibration ranges based on various considerations, including the size and shape of the skin treatment device and the desired vibration strength of the device. Examples of suitable ranges for vibration frequencies include vibration frequencies between about 20 Hz and about 500 Hz, vibration frequencies between about 100 Hz and about 400 Hz, and vibration frequencies between about 200 Hz and about 300 Hz. In another embodiment, the skin treatment device may comprise two or more motors. In another embodiment, the motor may be stochastic.

The pulsations provide a tapping motion to the skin, in some embodiments, by providing impulses to the skin's surface from the silicone touch-points (described below) pulsating against the skin's surface. The tapping provides a deeper clean that is less abrasive than scrubbing the skin with harsher bristles of other materials (e.g., nylon brush bristles) and provides deep facial cleansing of oil and dirt, helping to unclog follicles. The tapping motion can be generated by the vibrations of the motor or of multiple motors, or by electromechanical mechanisms, among other means. For example, motors can be positioned in the device to cause the vertical oscillations of the touch-points, such as by positioning one or more motors directly under or adjacent to the textured surface of the device. The oscillation of the motor(s) can cause each of or at least some of the touch-points to move orthogonal to the skin's surface to tap the skin. Multiple motors can be arranged near the textured surface to create different motions or different speeds of vertical oscillations across the textured surface of the skin. For example, the motors can be positioned so that different touch-point arrangements or patterns on the textured surface can oscillate differently from one another to provide one type of tapping motion for some touch-points and a different type (e.g., different speed, pattern, etc.) for other touch-points. In some embodiments, each touch-point is a single structure rather than a plurality of structures, such as might be found in a device where each bristle is made up of multiple components arranged in a pattern.

The massaging vibrations produced by the device can affect a number of positive results aside from cleansing, as well. For example, massaging the face and skin at particular frequencies aids in the stimulation of blood circulation and increases lymph flow. Such massaging can result in stimulation of collagen synthesis in fibroblast cells and improvement of muscle tone; this can result in healthier, younger-looking skin, such as in the known problem areas of the face, and a more sharply defined profile of the face. In particular, when gently applied to expression-line problem areas at the brow, temples and nasolabial folds, the skin treatment device can relax underlying muscles and reduce the appearance of dynamic wrinkles when such frequencies are used.

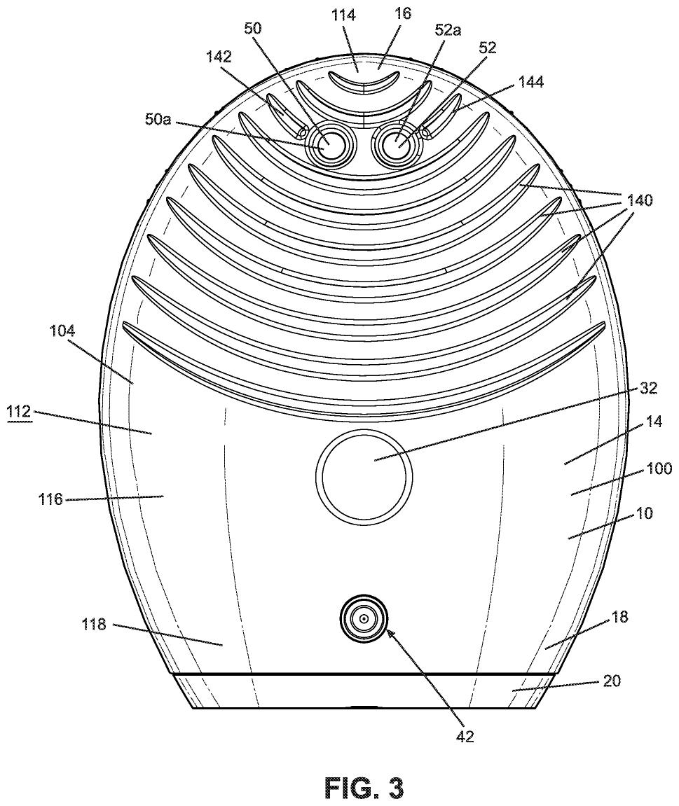

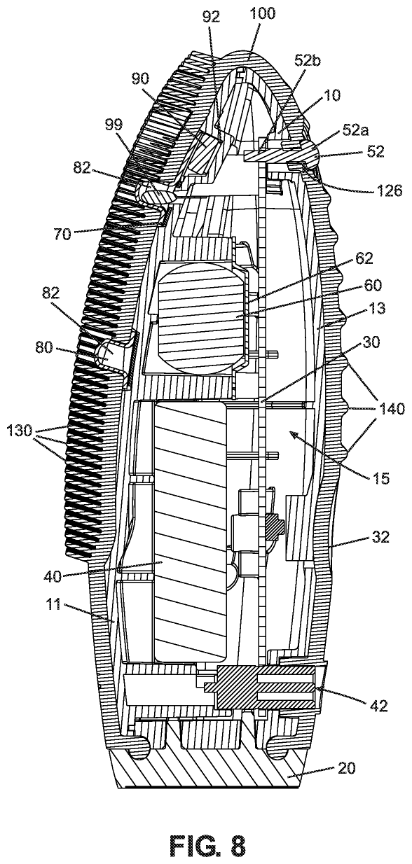

As noted above, the power source 40 is also disposed within the cavity 15 of the main body 10. The power source 40 is operatively connected to and provides power to the various components of the skin treatment device 2 so that they may function efficiently and properly. Such components include but are not limited to: the controller 30, the first and second stimulation mechanisms 50, 52, the motor 60, and the heating element 70. The power source 40 comprises a rechargeable lithium-ion battery in the illustrated embodiment, which may be charged through a charging port such as DC jack 42 shown in FIGS. 3 and 8. The power source 40 is housed substantially adjacent the first side 12 within the cavity 15. A skilled artisan will be able to select a suitable power source according to a particular example based on various considerations, including the various components housed within the skin treatment device and the desired dimensions of the skin treatment device. In other embodiments, the power source may comprise one, two, three, or more than three replaceable dry cell batteries. In different embodiments, the power source may comprise a lithium polymer battery or batteries.

The main body 10 also houses a controller 30, which is disposed within the main body 10 such that it extends along the length of a portion of the main body 10. The controller 30 is operatively connected to the first and second stimulation mechanisms 50, 52, the motor 60, the power source 40, and the heating element 70, and provides instructions to and controls each of these components. The controller 30 comprises a printed circuit board assembly (“PCBA”) and related circuitry; however, in other embodiments, the controller may comprise any device suitable to control the skin treatment device's components, such as a printed circuit board (“PCB”) or similar component.

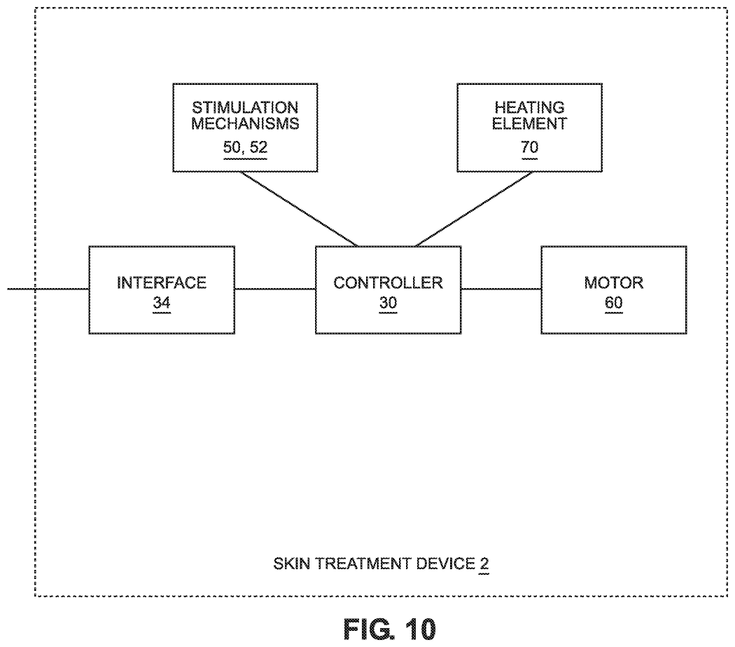

Additionally, the controller 30 comprises an interface 34 which allows it to communicate with a second device (see FIG. 10). The interface 34 allows the skin treatment device 2 to communicate with a second device, such as a personal computer, tablet, mobile telephone, or other electronic device (not illustrated in the Figures). Using the interface 34, the skin treatment device 2 can send information to other devices so that the other device(s) may collect data pertaining to the use of the skin treatment device 2. Furthermore, the skin treatment device 2 may receive control signals from another device that can indicate that the skin treatment device 2 should turn on or off, increase or decrease speed, use a different vibration, heating, or electrical stimulation pattern, and/or activate to a pre-set pattern desired by the user or recommended by the other device, among other instructions. The interface 34 can be a wired or a wireless interface, such as a wireless transceiver that transmits control signals between the skin treatment device 2 and the second device. A skilled artisan will be able to select a suitable interface based on various considerations, including the device with which the skin treatment device will communicate and the size and shape of the main body. In some embodiments, the interface is a radio-frequency (“RF”) transceiver used to transmit and receive RF signals between the skin treatment device and other devices. One example of an RF transceiver that could be used is a low power 2.4 GHz RF transceiver. In various embodiments, the skin treatment device may also include antennas for transmitting and receiving signals between the skin treatment device and other devices. In such examples, the interface can use BLUETOOTH®, Wi-Fi, infrared, laser light, visible light, acoustic energy, or one of a number of other methods to transmit information wirelessly between the skin treatment device and another device. Example data that the skin treatment device may communicate to one or more of a personal computer, tablet, mobile phone, or other electronic device may include the number of uses of the skin treatment device, the durations of the various uses of the skin treatment device, the user's preferred skin treatment device settings, and various other types of information related to the use of the skin treatment device.

In some embodiments, the skin treatment device is connected to a network via the second device. In other embodiments, the skin treatment device is directly connected to a wireless router or cellular phone network and may connect with the second device via any of the aforementioned means. Accordingly, the skin treatment device can be controlled via personal computer, tablet, mobile phone, or other suitable electronic devices a user using the personal computer, tablet, phone, or other device.

A user control 32 is disposed on the second side 14 of the skin treatment device 2 and is configured to allow a user to control one or more aspects of the device 2. The user control 32 is operatively connected to the controller 32 and, upon activation of the user control 32, the controller 30 will instruct the device 2 to perform one or more functions and/or actions. The user control 32 comprises a button in the illustrated embodiment, which allows for a user to activate the skin treatment device 2 by pressing the button. The user control 32 is disposed such that a user can easily press it while the device 2 is in use and held by a user. Optionally, repeated activation of the user control 32 allows for a user to cycle through various modes of operation of the skin treatment device 2. In various embodiments, potential modes that may be activated by the user control 32 include low frequency vibration mode, high frequency vibration mode, stochastic vibration mode, alternating frequency vibration mode, heat therapy mode, electrical current stimulation mode, and/or some combination thereof. A skilled artisan will be able to suitably place the user control on the device and configure the same according to a particular example based on various considerations, including the number of desired modes and the desired placement of the touch-points of the silicone exterior covering. In other embodiments, the user control may be disposed on the front of the main body, the side(s) of the main body, or the base of the main body. In a different embodiment, the skin treatment device may comprise zero, one, three, or more than three user controls. In additional embodiments, the user control may include a first user control to increase frequency of the motor and a second user control to decrease frequency of the motor.

The exterior covering 100 is illustrated in each of FIGS. 1 through 9. The exterior covering 100 comprises a single, unitary piece in this embodiment. The exterior covering 100 can be comprised of various elastic materials, preferably materials that are soft and do not damage the skin, such as silicone. Silicone is used as an example throughout, though it is understood that other materials can be used, as well. The exterior covering 100 is made of a hygienic silicone that is fast-drying and non-absorbent, allowing the skin treatment device 2 to be used with many skin cleansing products without wear. It is also configured to withstand temperature increases affected by the heating element 70 and electrical currents transferred by the stimulation mechanisms 50, 52. The exterior covering 100 includes a front surface 110 on a first exterior covering side 102 and a back surface 112 on a second exterior covering side 104 having varying textures. The exterior covering 100 also includes an upper portion 114, a middle portion 116, and a lower portion 118 and may define various structures on said front and back surfaces 110, 112.

The exterior covering 100 defines a set of passageways 120 (including passageways 120a, 120b, 120c, 120d, 120e, 120f, 120g, and 120h, hereinafter collectively referred to as “the passageways 120”) through which portions of the heat transfer mechanism 80 extends (described in greater detail below). Each of the passageways 120 is substantially cylindrical in shape. The passageways 120 are aligned such that a substantially circular portion 122 of the front surface 110 is disposed between and cooperatively defined by the passageways 120. The first passageway 120a is substantially opposite the fifth passageway 120e about the substantially circular portion 122. The second passageway 120b is substantially opposite the sixth passageway 120f about the substantially circular portion 122. The third passageway 120c is substantially opposite the seventh passageway 120g about the substantially circular portion 122. The fourth passageway 120d is substantially opposite the eighth passageway 120h about the substantially circular portion 122. Each of the passageways 120 may have any suitable diameter. A skilled artisan will be able to select a suitable number of passageways and their sizes and formations according to a particular example based on various considerations, including the size and shape of the skin treatment device and the functionalities of the same. In other embodiments, the skin treatment device may include zero, one, two, three, four, five, six, seven, nine, or more than nine passageways. In different embodiments, each of the passageways may have any shape, including that of a pill, cylinder, ellipse, oval, triangle, square, and/or rectangle; in such embodiments, one passageway may have a different shape than any other passageway. They can also be aligned in any configuration. Suitable example passageway diameters include diameters between about 2 millimeters (“mm”) and about 50 mm, diameters between about 5 mm and about 25 mm, and diameters between about 10 mm and about 20 mm.

The front surface 110 also defines a set of touch-points 130 for gentle cleansing of the user's skin. The touch-points 130 comprise a portion of the textured surface of the exterior covering 100. The touch-points 130 extend from the upper portion 114 towards the base 20 of the skin treatment device 2. The touch-points 130 are disposed on both the upper portion 114 and middle portion 116. In the illustrated embodiment, the touch-points 130 each have substantially the same shape, diameter, and height. In different embodiments (and as described below), the touch-points may have different shapes, diameters, heights, and alignments. The touch-points 130 are compressible and bendable such that they conform to the surface of the skin during use. A skilled artisan will be able to determine how to suitably shape, size, and place the various touch-points according to a particular example based on various considerations, including the skin type of potential users and the size and shape of the skin treatment device. In various embodiments, the touch-points may be between about 0.5 mm in diameter and about 8 mm in diameter, between about 1.5 mm in diameter and about 5 mm in diameter, and between about 2 mm in diameter and about 3 mm in diameter. The textured portion of the exterior covering can comprise between about 5% and about 80% of the exterior covering, between about 20% and about 60% of the exterior covering, and between about 30% and about 50% of the exterior covering according to various embodiments. The textured portion can be positioned on an upper portion or tip of the device, such as is shown in the FIGS., but can also be otherwise positioned (e.g., at the sides, in the middle, at the bottom, etc.). Thus, the touch-points can also be arranged differently around the front surface than is illustrated. In other embodiments, the textured surface can be designed to pivot relative to the main body or to include one or more portions that pivot such that the textured surface can mold to the shape of the face. In some embodiments, the exterior covering may be comprised of many materials, including any suitable elastomeric material. In different embodiments, the exterior covering can be made of multiple pieces, rather than a single unitary piece.

Additionally, different types of touch-points can be included, such as taller or shorter touch-points, touch-points with more or less bulbous ends, touch-points with ends of different shapes (e.g., pointed, feathered, ridged, etc.) and/or diameters, and so forth. Similarly, the touch-points can be arranged more or less densely, can be positioned on both the front and back of the device, and can be otherwise located on the device (e.g., only in the middle, only at the edges, etc.), among other variations. The above are just some examples of different device surface configurations matched to different skin types. Other touch-point alignments for other skin types are also possible, such as a particular pattern for dry skin, for aging skin, for combination skin or T-zone skin (e.g., oilier around the forehead, nose, and chin), among others. In some embodiments, the front textured surface includes at least two different types of touch-points (e.g., thicker and thinner). The touch-points of a type can be grouped to provide a pattern. Various patterns can correspond to a particular skin type (e.g., male, sensitive, oily, normal, etc.).

The back surface 112 also includes a textured surface area having touch-points 140. The touch-points 140 on the back surface 112, however, are in the form of raised ridges which protrude away from the front surface 110 of the exterior covering 100. The touch-points 140 are arranged in a wave formation and intended to provide minimal abrasion and maximal pulsation energy transfer. The touch-points 140 are substantially arcuate in shape, though those touch-points 140 which are closer to the user control 32 are longer than those disposed adjacent the upper portion 114 of the exterior covering 100. In the illustrated embodiment, there are a total of nine ridged touch-points 140; there are also two portions 142, 144 of a ridge-shaped touch-point 140 that are interrupted by the first and second stimulation mechanisms 50, 52. These touch-points 140 can contact a user's skin during anti-aging routines. They transfer massage, heat, and/or electrical current stimulation therapy to the user.

A skilled artisan will be able to determine how to suitably shape, size, and place the touch-points disposed on the back surface according to a particular example based on various considerations, including the skin type of potential users and the size and shape of the skin treatment device. The textured portion of the exterior covering having the back surface touch-points can comprise between about 5% and about 80% of the exterior covering, between about 20% and about 60% of the exterior covering, and between about 30% and about 50% of the exterior covering according to various embodiments. The textured portion can be positioned on an upper portion or tip of the device, such as is shown in the FIGS., but can also be otherwise positioned (e.g., at the sides, in the middle, at the bottom, etc.). Thus, the touch-points can also be arranged differently around the back surface than is illustrated. In different embodiments, the back surface may define any number of touch-points, including one, two, three, four, or more than four. Additionally, the back surface may define a set of touch-points that are similar to those of the front surface, rather than ridge-shaped touch-points.

The back surface 112 also defines two rear passageways 124, 126 that are configured to allow the first and second stimulation mechanisms 50, 52 to protrude through the exterior covering 100 such that a user may contact said stimulation mechanisms. The rear passageways 124, 126 are substantially cylindrical in shape in the illustrated embodiment. However, in other embodiments, they may have any shape. A skilled artisan will be able to select a suitable number of rear passageways and their sizes and alignments according to a particular example based on various considerations, including the size and shape of the skin treatment device and the functionalities of the same. In other embodiments, the skin treatment device may include zero, one, three, four, or more than four passageways. In different embodiments, each of the rear passageways may have any shape, including that of a circle, cylinder, ellipse, oval, triangle, square, and/or rectangle; in such embodiments, one passageway may have a different shape than any other passageway. Suitable example passageway diameters include diameters between about 2 millimeters (“mm”) and about 50 mm, diameters between about 5 mm and about 25 mm, and diameters between about 10 mm and about 20 mm.

The heating element 70 is substantially disc-shaped and defines a first passageway 72 and a second passageway 74. Each of the first and second passageways 72, 74 is substantially cylindrical in shape and extends from the first surface 71 to the second surface (not illustrated in the Figures) of the heating element 70. Each of the first and second passageways 72, 74 are uniform along their length; the first passageway 72 has a larger diameter than does the second passageway 74. The first passageway 72 is configured to be disposed about the protrusion 24 of the indented portion 22 of the first piece 11. Accordingly, a substantial portion of the body of the heating element 70 is disposed within the indented portion 22. The heating element 70 is configured to snugly fit within the indented portion 22 such that it does not become dislodged as the user tilts, moves, uses, and otherwise manipulates the device 2.

The second passageway 74 is configured such that it may align with crevice 29 defined by the indented portion 22. The crevice 29 and second passageway 74 are, essentially, aligned in such a way that they continuously form a single passageway configured to allow a pin or similar mechanical device to be inserted into the passageway that would aid in holding the heating element 70 in place within the indented portion 22 and/or provide another functionality, as described below.

The heating element 70 may be comprised of aluminum, for example, and is operatively connected to each of the power source 40 and the controller 30. The heating element 70 is capable of generating heat when instructed to do so via the controller 30. The heat is then distributed to the heat transfer mechanism 80 and, indirectly, to the exterior covering 100 so that a user is able to receive heat therapy when he or she desires such therapy from the device 2.

A skilled artisan will be able to select and configure a suitable heating element according to a particular example based on various considerations, including the shape and size of the heat transfer mechanism and the shape and size of the main body. In other embodiments, the heating element may be comprised of aluminum, aluminum alloys, other metals and/or metal alloys, copper, stainless steel, ceramic, and/or any other suitable material. In different embodiments, the heating element and/or its passageways may have any shape, including that of an oval, square, rectangle, triangle, ellipse, and/or pill. In additional embodiments, the heating element may have zero, one, three, or more than three passageways (each of which being capable of having any shape) and may be secured to the first piece via any mechanism, including one or more snap-fit or other mechanical mechanisms or one or more adhesives. Additionally, the indented portion into which the heating element is placed may differ in shape, size, and/or configuration. In various embodiments, the heating element be heated to temperatures between about 70 degrees Fahrenheit (“° F.”) and about 150° F., between about 75° F. and about 125° F., and between about 80° F. and about 100° F. In some embodiments, the heating element may include one or both of an N-type semiconductor and/or a P-type semiconductor.

The heat transfer mechanism 80 may be comprised of any suitable material, including aluminum, and is configured to be attached to the first piece 11 adjacent the heating element 70. The heat transfer mechanism 80 includes an attachment mechanism 84 at its proximal end.

The heat transfer mechanism 80 includes a front surface 81 that is substantially flat, as well as eight nodes 82 (including first node 82a, second node 82b, third node 82c, fourth node 82d, fifth node 82e, sixth node 82f, seventh node 82g, and eighth node 82h). Each of the nodes 82 protrudes from the front surface 81 of the heat transfer mechanism 80 and includes a substantially flat end. The nodes 82 are configured such that they may extend through the exterior covering's 100 passageways 120 (including passageways 120a, 120b, 120c, 120d, 120e, 120f, 120g, and 120h). More specifically, the first node 82a extends through the first passageway 120a, the second node 82b extends through the second passageway 120b, the third node 82c extends through the third passageway 120c, and so forth. The nodes 82 are aligned in a substantially circular formation. Each of the nodes 82 extends through its respective passageway 120 to such a degree that a user could contact the end of each node 82 if he or she so desired. The nodes 82 do not, however, extend further than the tip of any touch-point of the set of touch-points 130 defined by the first exterior covering side 102 in the illustrated embodiment.

The heat transfer mechanism 80 also defines a passageway 83 extending from its front surface 81 to its rear surface (not illustrated in the Figures). The passageway 83 is substantially cylindrical in shape and is uniform in diameter along its length. The passageway 83 is configured to be disposed about the protrusion 24 of the first piece 11 and disposed adjacent the heating element 70 once both have been secured to the first piece 1. At least a portion of the heat transfer mechanism 80 shall be disposed within or adjacent the indented portion 22. In fact, in the illustrated embodiment the heat transfer mechanism 80 is configured to snugly fit within the indented portion 22 such that it does not become dislodged as the user tilts, moves, uses, and otherwise manipulates the device 2.

The attachment mechanism 84 of the heat transfer mechanism 80 provides a second mechanism through which the heat transfer mechanism 80 may be secured to the first piece 11. More specifically, the attachment mechanism 84 is configured such that it may be secured to the attachment portion 25 of the first piece 11 via a snap-fit mechanism. One end of the attachment mechanism 84 includes an extension 85 configured to be inserted into the cutout 25a of the attachment portion 25. Two protrusions 86a, 86b also extend away from the center of the heat transfer mechanism 80. The protrusions 86a, 86b are configured to snap-fit into the cutouts 28a, 28b of the indented portion 22.

The heat transfer mechanism 80 absorbs heat that is released by the heating element 70. The heat transfer mechanism 80 then transfers said heat to the user when the device 2 is in use via either direct (such as through the nodes 82) or indirect (such as through a portion of the exterior covering 100) contact by the user. The heat transferred to the user provides the basis for the heat therapies described herein.

The device 2 is purposely configured such that the heat transfer mechanism 80 is placed above the heating element 70 and between heating element 70 and exterior covering 100. This configuration allows it to both transfer heat to the user and shield the exterior covering 100 from direct contact with the heating element 70. Such a configuration ensures that the heating element 70 does not directly heat the exterior covering 100, which could negatively impact the exterior covering 100 if done improperly. In other embodiments, the heating element and heat transfer mechanism are combined into a single unit.

A skilled artisan will be able to select a suitable heat transfer mechanism according to a particular example based on various considerations, including the size and shape of the heating element and the desired heating capability of the device. In various embodiments, the heat transfer mechanism may include zero, one, two, three, four, five, six, seven, nine, or more than nine nodes. In different embodiments, the nodes may have any shape and size and may be disposed in any formation. In other embodiments, the nodes may extend past or on the same plane as touch-points. In alternate embodiments, the attachment mechanism may have any shape and size and can be attached to any portion of the first piece via any suitable mechanism. In yet other embodiments, the heat transfer mechanism may be omitted altogether. In additional embodiments, the heat transfer mechanism may be disposed on any portion of the first piece; it need not be disposed adjacent and/or above the heating element. In various embodiments, the heat transfer mechanism may be comprised of any suitable material(s), including aluminum, aluminum alloys, stainless steel, metals, metal alloys, and/or ceramic.

A temperature sensor 99 is also disposed within the main body 10. The sensor 99 is substantially pill-shaped and includes a tail. The sensor 99 may be operatively connected to one or both of the power source 40 and controller 30 and is configured to measure the temperature of the skin treatment device 2 and report said temperature to the controller 30. If the sensor 99 detects that the temperature of the skin treatment device 2 passes a certain threshold, it will transmit said information to the controller 30 and the controller 30 will initiate a “shut down” of the device in order to protect its user from the potential effects of an overheated device (such as burning the skin, for example). In such instances, the skin treatment device 2 will cease operating; such a cessation of operation may be permanent or temporary. In the illustrated embodiment, the sensor 99 comprises a negative temperature coefficient thermistor; however, in other embodiments, the sensor may be comprised of any suitable component. A skilled artisan will be able to a select suitable sensor according to a particular example based on various considerations, including the specific temperature control unit used and the size and shape of the device. In different embodiments, the sensor may be comprised of any suitable material, including various semiconductors, copper, and other suitable materials. In yet other embodiments, the skin treatment device may include zero, two, three, or more than three sensors. In various examples, the sensor may instruct the controller to cease operation of the device when the sensor detects a temperature that exceeds about 120° F., about 150° F., and/or about 180° F.

Moreover, a thermal fuse 90 may be used along with or in place of the sensor 99. The thermal fuse 90 is disposed within the cavity 15 and, in the event it detects that the device 2 has exceeded a particular temperature threshold, will activate to prevent the device 2 from continuing to heat. Thermal fuses are generally known in the art and many commercially available thermal fuses could comprise thermal fuse 90. The thermal fuse 90 is disposed adjacent a silicone support mechanism 92 and held in place within the main body 10 by the same. In other embodiments, more than one thermal fuse may be used or the thermal fuse may be omitted.

The skin treatment device 2 also includes a first stimulation mechanism 50 and a second stimulation mechanism 52. The first and second stimulation mechanisms 50, 52 (collectively, “the stimulation mechanisms”) are partially disposed within the cavity 15 of the main body 10 and partially extend through the second side 14 and exterior covering 100 such that a user may directly contact the first and second stimulation mechanisms 50, 52. They generally are disposed adjacent the upper portion 16 of the second side 14; relatively speaking, they are disposed closer to the upper portion 16 than to the base 20. Each of the stimulation mechanisms 50, 52 include first ends (50a and 52a, respectively) that are substantially rounded and second ends (50b and 52b, respectively) that are elongate and have a smaller diameter than do the first ends 50a, 52a. More specifically, the first ends 50a, 52b extend through the rear passageways 124, 126 to such an extent that they may be contacted by a user. This allows for a user to obtain the full benefit of the stimulation mechanisms' functions (as described below). The second ends 50h, 52b extend into the cavity 15 such that portions of the stimulation mechanisms 50, 52 may be secured in place within the main body 10.

The stimulation mechanisms 50, 52 are comprised of one or more metals, such as aluminum in some examples, and are operatively connected to each of the power source 40 and the controller 30. The stimulation mechanisms 50, 52 are capable of transferring electrical current to the user when instructed to do so via the controller 30. Specifically, the stimulation mechanisms 50, 52 transfer electrical currents to the skin, muscles, tissue, and other cells of the user. Contact with the skin of the user allows such a current to be transferred; this can allow the device 2 to provide one or more of the positive results described above. Such electrical current stimulation is typically referred to as “micro-current stimulation” in the beauty and cosmetic industries and devices which transfer the same to a user for cosmetic and/or beauty applications are known as “micro-current devices.” In various embodiments, the electrical current therapy transferred to a user via the stimulation mechanisms may have a voltage between about 10 volts (“V”) and about 200V, between about 30V and about 70V, and between about 40V and about 60V. In a different embodiment, the electrical current therapy transferred to a user via the stimulation mechanisms may

A skilled artisan will be able to select suitable stimulation mechanism(s) according to a particular example based on various considerations, including the size and shape of the skin treatment device and the sizes and shapes of the stimulation mechanism(s). In one embodiment, the skin treatment device may include zero, one, three, or more than three stimulation mechanisms. In a different embodiment, the stimulation mechanisms may have first and second ends having any shape, including that of a cylinder, ellipse, pyramid, box, and/or pill. The first stimulation mechanism may be shaped differently than the second stimulation mechanism in some embodiments. One or more stimulation mechanisms may be comprised on the first side of the device, as well. In additional embodiments, one or both stimulation mechanisms may extend past one or more touch-points of the second side, such that they are on the same plane as one or more touch-points of the second side, and/or short of one or more touch-points of the second side. The stimulation mechanisms may also be disposed on any portion of the second side. In some embodiments, one or both of the stimulation mechanisms may have a positive end and a negative end. In different embodiments, one or both of the stimulation mechanisms may be used to wirelessly charge and/or provide power to the power source. In other embodiments, a serum, cream, lotion, gel, or other cosmetic preparation or material may be placed on a user's skin prior to the activation of the stimulation mechanisms.

In use, a user will place the skin treatment device 2 in his or her hand. As described above, the hand may be splayed about the second side 14 of the device 2. The user can then activate the device 2 via either the user control 32 or a second device, such as a mobile phone having an application that includes software and is compatible with the device 2. The user can then select a suitable treatment via one or more of the user control 32 or, in the example provided above, the mobile phone application. The device 2 can then provide one or more of massage therapy, heat therapy, and/or electrical current stimulation. While one or more such therapies are being provided, the user will place the device 2 to his or her face, neck, or other desired portion of the body. The device 2 will then provide the selected treatment patterns and/or therapies to the user. If the device 2 is connected to a mobile phone or similar second device, the mobile phone application can track the user's usage data, including: time spent using the device 2, which treatment(s) were selected, time and date of usage, and similar information. The application can then recommend other treatments and/or how to improve selected treatments.

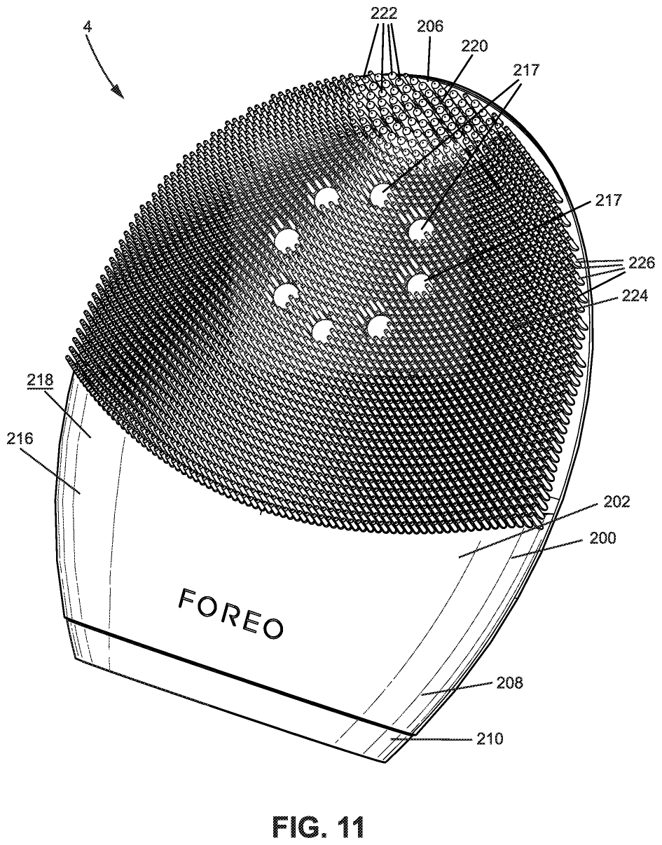

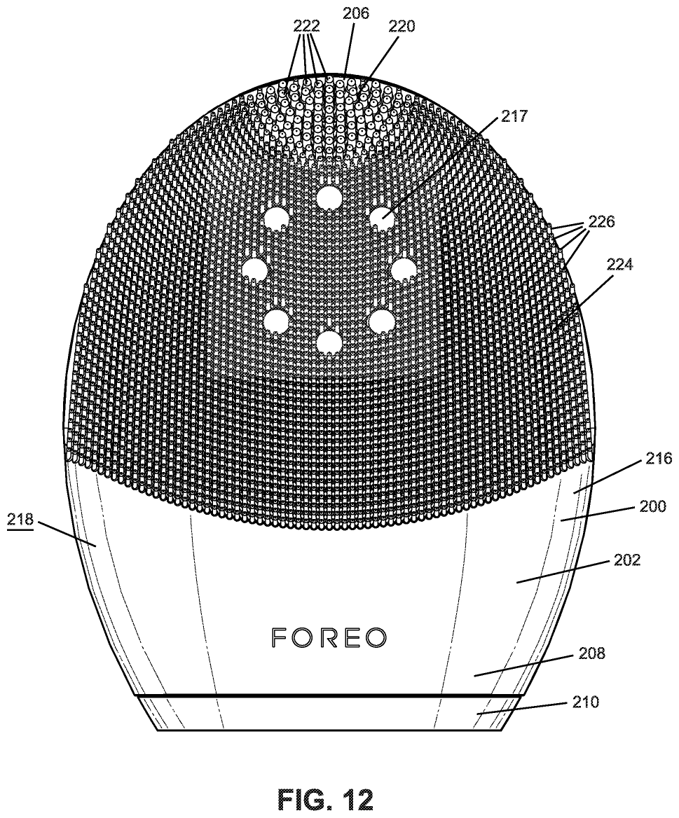

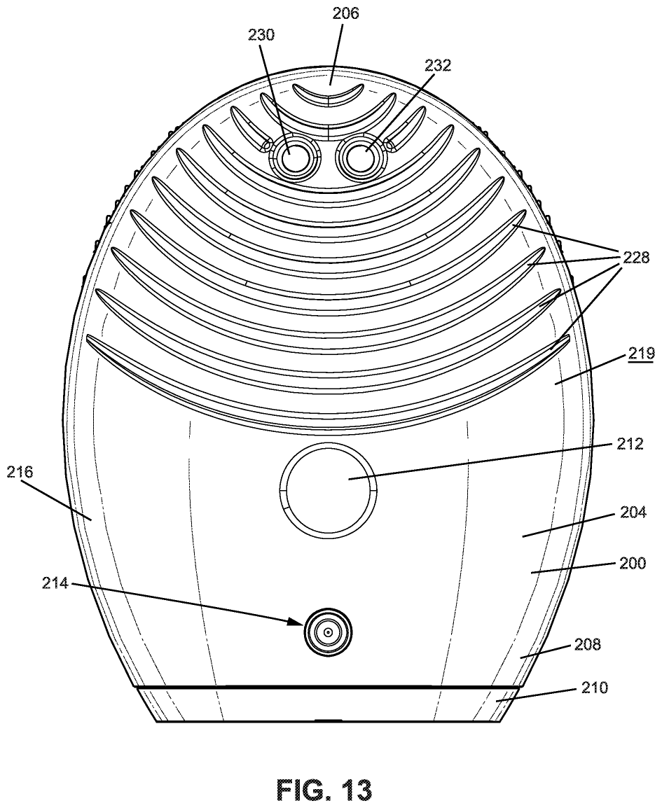

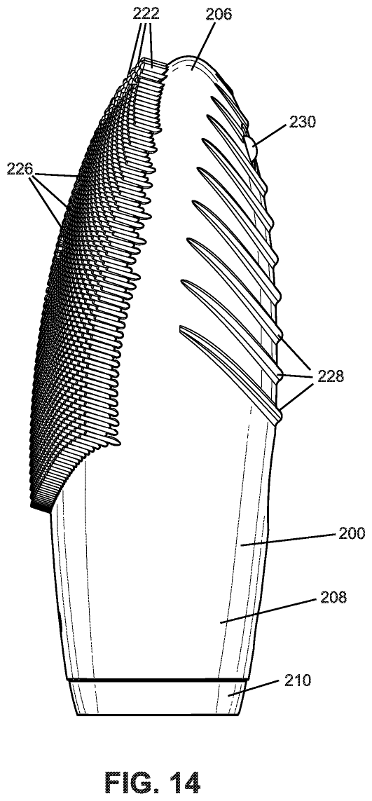

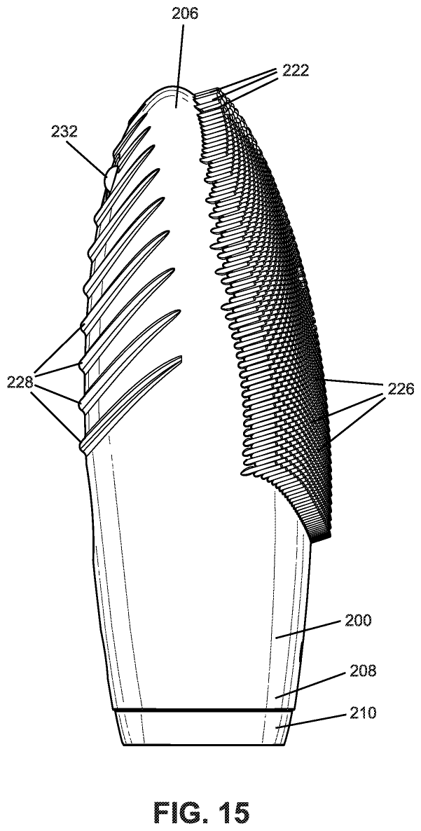

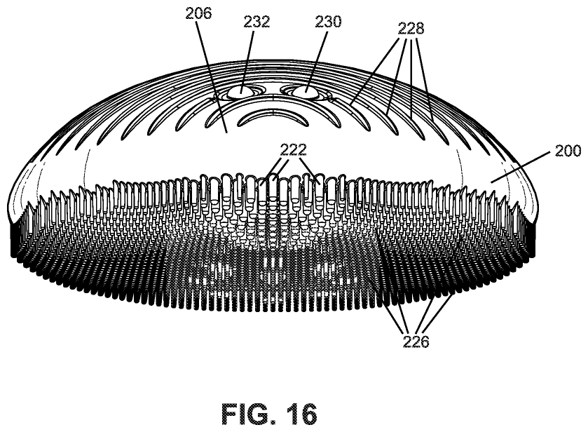

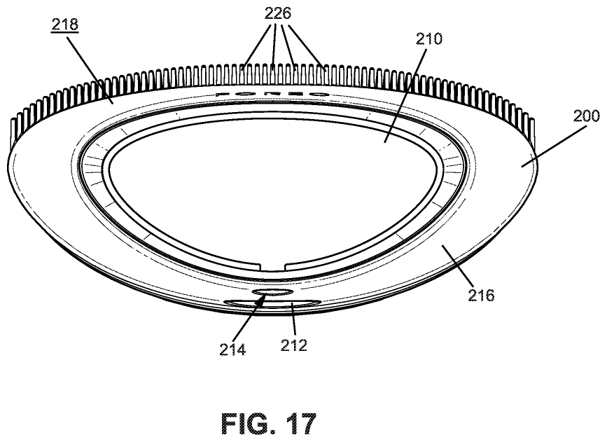

FIGS. 11 through 17 illustrate another example skin treatment device 4 and/or one or more components thereof. The skin treatment device 4 is similar to the skin treatment device 2 shown in FIGS. 1 through 9, except as described below. Thus, the skin treatment device 4 comprises at least a main body 200 having a first side 202, a second side 204, an upper portion 206, a lower portion 208, a base 210, a controller (not illustrated in the FIGS.), a motor (not illustrated in the FIGS.), a power source (not illustrated in the FIGS.), a user control 212, a charging port 214, an exterior covering 216, a heating element (not illustrated in the FIGS), a heat transfer mechanism 217, and first and second stimulation mechanisms 230, 232.

In the illustrated embodiment, the exterior covering 216 includes a first textured surface area 220 having a first set of touch-points 222 on the front surface 218 of the exterior covering 216 and a second textured surface area 224 having a second set of touch-points 226 on the front surface 218. The touch-points of the first set of touch-points 222 have a larger diameter than those touch-points of the second set of touch-points 226. Additionally, the touch-points of the first set of touch-points 222 are grouped adjacent one another within the first textured surface area 220, while the touch-points of the second set of touch-points 226 are disposed within the second textured surface area 224. The passageways (not illustrated in the embodiment) through which the nodes of the heat transfer mechanism 217 protrude and are able to be touched by the user are disposed within the second textured surface area 224. The touch-points of the first set of touch-points 222 can target oiler areas and hard-to-reach points around the nose, ears, and hairline. Their increased diameter (and, thus, resistance) helps in removing oil in such areas. The thinner touch-points of the second set of touch-points 226 can target other areas, including those which are hard to reach via the thicker touch-points of the first set of touch-points 222. Having touch-points of different diameters ensures that all of a user's skin can be reached by the skin treatment device 4. This pattern of touch-points is one example of a pattern that might be used to cleanse what is commonly referred to as “normal” skin. Normal skin can include some areas that are drier, oilier, or more sensitive, such that the different patterns of thinner and thicker touch-points can be helpful in targeting these areas. The thinner touch-points and thicker touch-points may vary in size and spacing in ways different than described above. In various embodiments, the touch-points of the second set of touch-points are 25-80% thinner (e.g., 30%, 40%, 50%, 60%, 70%, etc. or values in between) compared to the thicker touch-points. In various embodiments, the touch-points of the second set of touch-points are spaced closer together (i.e., the distance between touch-points) by 15%-60%, as compared with those of the first set of touch-points. Thus, the thinner and thicker touch-points for the normal skin cleansing can also be arranged differently than is illustrated. In one embodiment, the first set of touch-points are each between about 0.5 mm and 4.5 mm in diameter, and the second set of touch-points are each between about 0.2 mm and 2.5 mm in diameter. In some embodiments, a number of intermediate-sized touch-points (i.e., those having a diameter small than those of the first set of touch-points and larger than the diameters of those of the second set of touch-points) may be disposed on the exterior covering adjacent the first set of touch-points and the second set of touch-points. Additionally, while in the illustrated embodiment the touch-points 228 on the back surface 219 are in the form of raised ridges that are similar to those of skin treatment device 2, in other embodiments they may comprise bristles of any diameter or any suitable number of ridges having any shape and size.

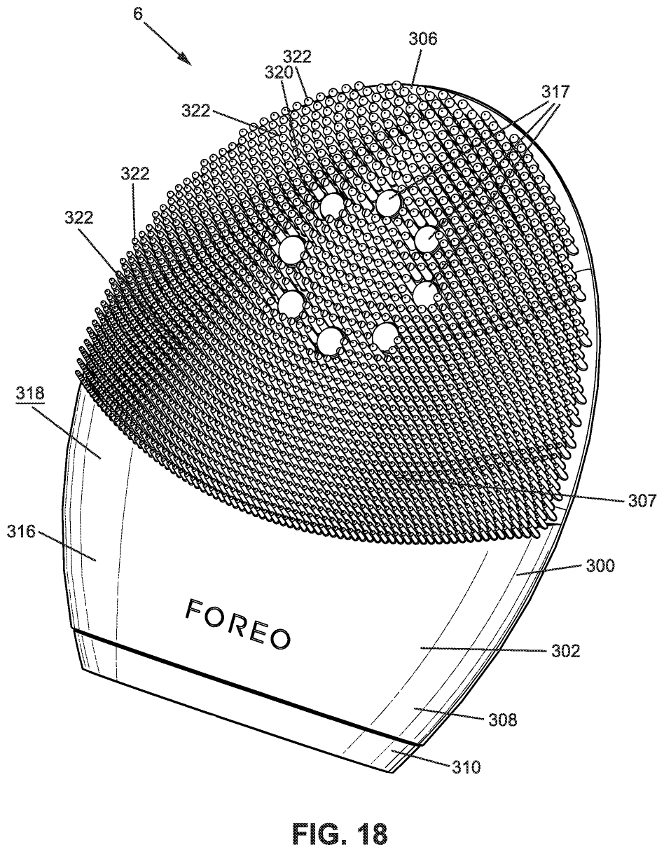

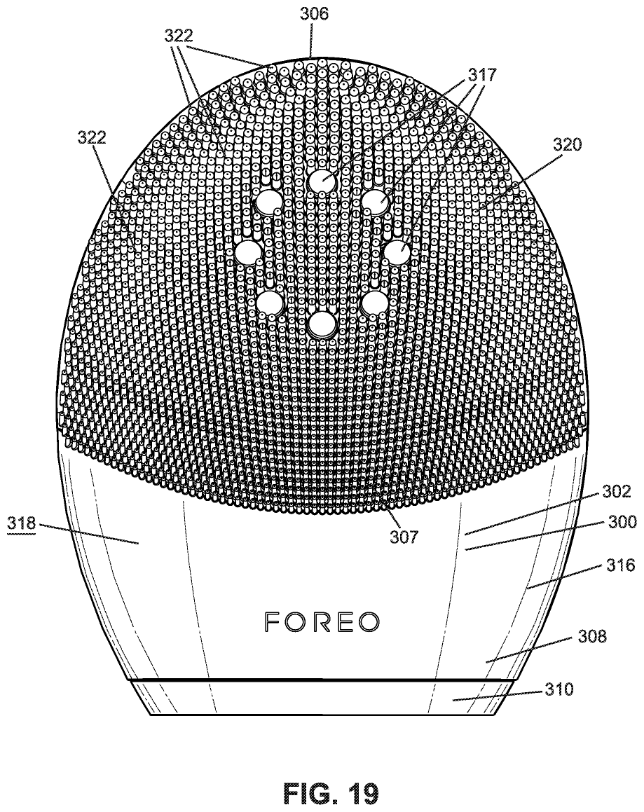

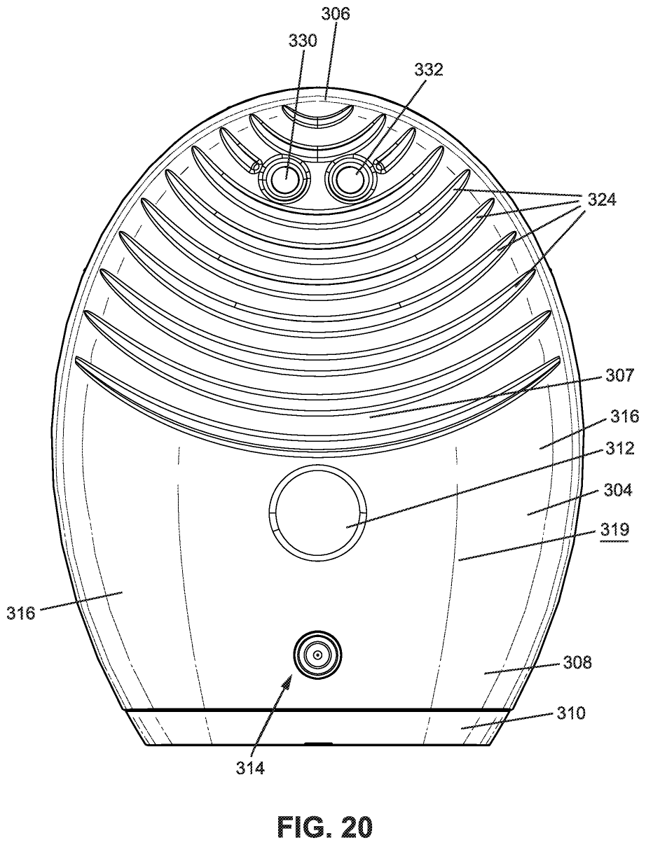

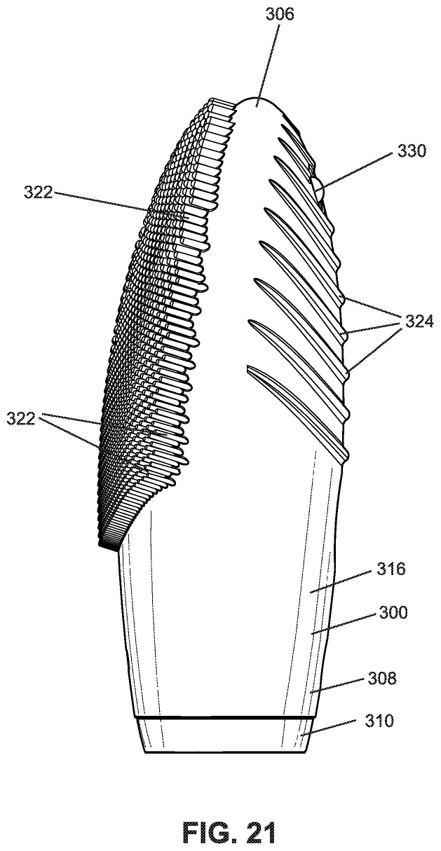

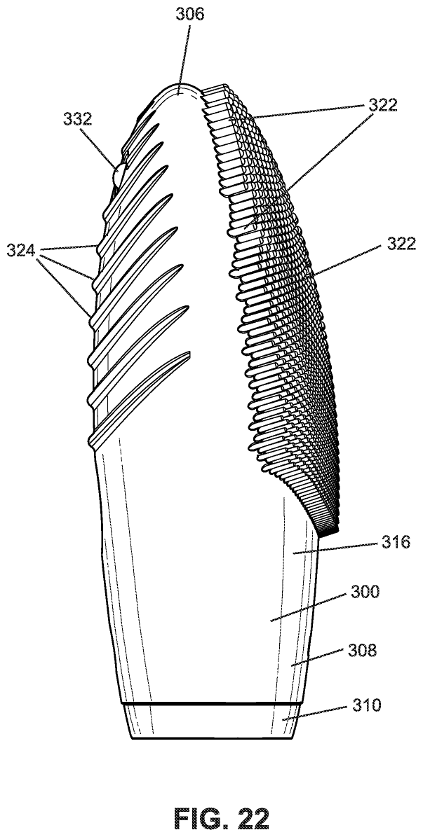

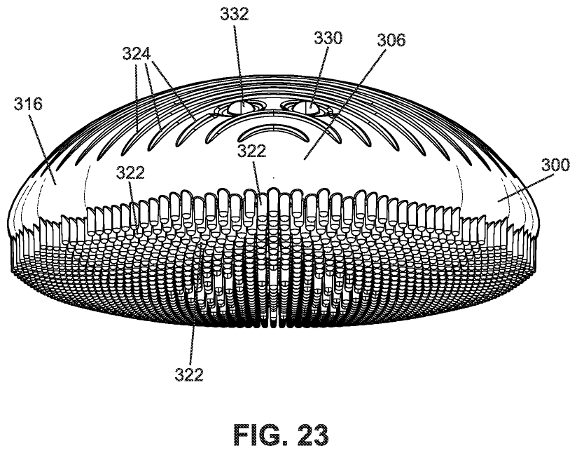

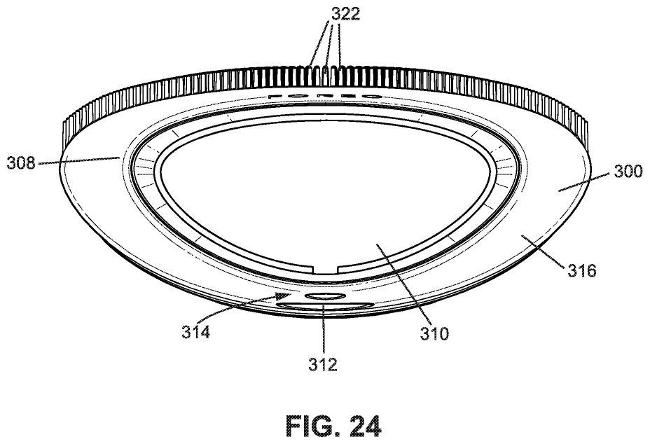

FIGS. 18 through 24 illustrate another example skin treatment device 6 and/or one or more components thereof. The skin treatment device 6 is similar to the skin treatment device 4 shown in FIGS. 11 through 17, except as described below. Thus, the skin treatment device 6 comprises at least a main body 300, a first side 302, a second side 304, an upper portion 306, a middle portion 307, a lower portion 308, a base 310, a controller (not illustrated in the FIGS.), a motor (not illustrated in the FIGS.), a power source (not illustrated in the FIGS.), a user control 312, a charging port 314, an exterior covering 316, a heating element (not illustrated in the FIGS), a heat transfer mechanism 317, and first and second stimulation mechanisms 330, 332.

In the illustrated embodiment, the textured surface area 320 includes a set of touch-points 322 disposed on the front surface 318 of the exterior covering 316 having varying diameters. Unlike the touch-points on skin treatment device 4, the touch-points of the set of touch-points 322 of the illustrated embodiment gradually decrease in size along the first side 302 of the device 6, rather than being disposed within two textured surface areas generally including thicker and thinner touch-points. More specifically, the textured surface area 320 generally includes thicker touch-points at or near the upper portion 306 of the device 6, while thinner touch-points are disposed nearer the lower portion 308 of the device 6 (relative to the position of the thicker touch-points). The set of touch-points 322, however, also includes a number of intermediate-sized touch-points that are disposed between the thinnest and the thickest touch-points of the set of touch-points 322. These also generally decrease in thickness along the front surface 318, with thicker touch-points disposed closer to the upper portion 306 and thinner touch-points being disposed closer to the lower portion 308. This configuration includes additional types of touch-points that may assist a user in ensuring that he or she is able to effectively and efficiently massage and/or cleanse his or her face. The touch-points may vary in size and spacing in ways different than described above. In various embodiments, the thinner touch-points are 25-80% thinner (e.g., 30%, 40%, 50%, 60%, 70%, etc. or values in between) than the thicker and/or intermediate touch-points. In various embodiments, the thinner touch-points are spaced closer together (i.e., the distance between touch-points) by 15%-60% than are the thicker and/or intermediate touch-points. In one embodiment, the thicker touch-points are between about 0.5 mm and 4.5 mm in diameter, and the thinner touch-points are between about 0.2 mm and 2.5 mm in diameter. In some embodiments, the intermediate-sized touch-points may include two, three, four, five, six, or more than six touch-points having different diameters in various embodiments. Additionally, while in the illustrated embodiment the touch-points 324 on the back surface 319 are in the form of raised ridges that are similar to those of skin treatment device 4, in other embodiments they may comprise bristles of any diameter or any suitable number of ridges having any shape and size.

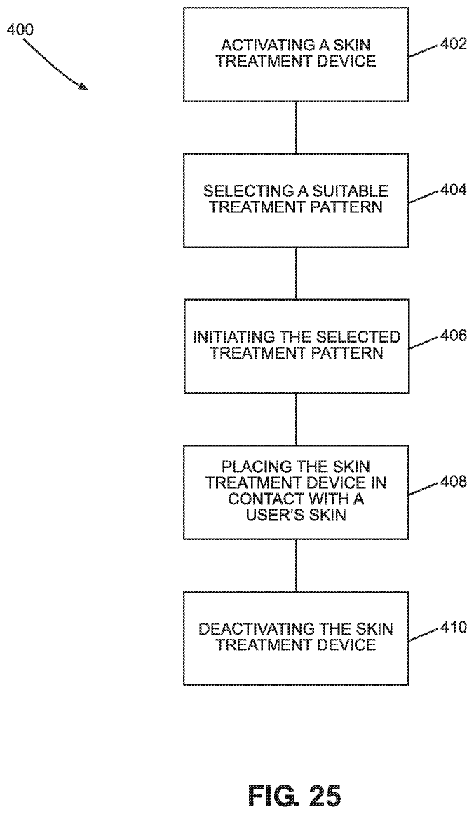

FIG. 25 is a flowchart representation of an example method 400 of using a skin treatment device. Performance of this method results in the treatment of the user's body, such as the face and/or neck, by a skin treatment device. This method can be used for treating any portion of a human body.

An initial step 402 comprises activating a skin treatment device, such as skin treatment device 2, by pressing a user control, such as user control 32, in order to ready the skin treatment device 2 for use. In other embodiments, any skin treatment device can be used, including skin treatment device 4 or skin treatment device 6.

Another step 404 comprises selecting a suitable treatment pattern. A treatment pattern may be selected via the user control 32 or via a second device that can communicate with the skin treatment device 2. A particular treatment pattern may include massage therapy, heat therapy, electrical current stimulation, and one or more of these.

Another step 406 comprises initiating the selected treatment pattern.

Another step 408 comprises placing the skin treatment device 2 in contact with or adjacent the user's skin. Doing so allows the skin treatment device 2 to treat the skin.

Another step 410 comprises deactivating the skin treatment device 2. Deactivation of the skin treatment device 2 may occur automatically at the end of a treatment pattern or via manipulation of the user control 32.

It is noted that it is advantageous to complete this method 400 in the order illustrated and described. However, any order is considered suitable.

In all examples, a skin treatment device and its various components may be formed of any suitable material, including presently known and later-developed materials. A skilled artisan will be able to select appropriate materials for an example skin treatment device based on various considerations, including the size and shape of the device, the motor housed within the device, the heat source housed within the device.

Those with ordinary skill in the art will appreciate that various modifications and alternatives for the described and illustrated embodiments can be developed in light of the overall teachings of the disclosure. Accordingly, the particular arrangements disclosed are intended to be illustrative only and not limiting as to the scope of the invention, which is to be given the full breadth of the appended claims and any and all equivalents thereof.

What is claimed is:

1. A skin treatment device having a main body configured to treat the skin of a user, comprising:
a substantially flat base configured to stand unaided on a substantially flat surface, said main body having a cross-sectional shape that is longer in a first direction substantially parallel to the base than in a second direction substantially parallel to the base;
a silicone exterior defining a first set of touch-points integrally formed with the silicone exterior, the first set of touch-points being disposed on a first side of said main body;
an oscillating motor disposed within said main body, the oscillating motor configured to produce pulsations of said skin treatment device;
a heating element disposed adjacent the first side of the main body, the heating element configured to produce heat; and
a heat transfer mechanism disposed adjacent the heating element and silicone exterior, the heat transfer mechanism configured to transfer the heat to said skin.

2. The skin treatment device of claim 1, wherein the main body defines an indented portion and a protrusion extending away from a surface of the indented portion.

3. The skin treatment device of claim 2, wherein the heating element is at least partially disposed within the indented portion and adjacent the protrusion.

4. The skin treatment device of claim 3, wherein the heating element is comprised of one or more metals.

5. The skin treatment device of claim 4, wherein the heat transfer mechanism is at least partially disposed within the indented portion and adjacent the protrusion; and
wherein the heat transfer mechanism is disposed above the heating element within the indented portion.

6. The skin treatment device of claim 5, wherein the heat transfer mechanism is comprised of one or more metals and defines a set of nodes.

7. The skin treatment device of claim 6, wherein the exterior covering defines a set of passageways through which at least portions of the nodes of the set of nodes extend.

8. The skin treatment device of claim 7, wherein the nodes are configured to be contacted by a user to transfer heat therapy to the user when said skin treatment device is in use.

9. A skin treatment device having a main body configured to treat the skin of a user, comprising:
a substantially flat base configured to stand unaided on a substantially flat surface, said main body having a cross-sectional shape that is longer in a first direction substantially parallel to the base than in a second direction substantially parallel to the base;
a silicone exterior defining a first set of touch-points integrally formed with the silicone exterior, the first set of touch-points being disposed on a first side of said main body;
an oscillating motor disposed within said main body, the oscillating motor configured to produce pulsations of said skin treatment device; and
a stimulation mechanism disposed on a second side of said main body, the stimulation mechanism configured to transfer electrical current stimulation to said skin.

10. The skin treatment device of claim 9, wherein the stimulation mechanism is comprised of one or more metals.

11. The skin treatment device of claim 10, wherein the electrical current stimulation transferred by the stimulation mechanism is between about 50 volts and about 120 volts.

12. The skin treatment device of claim 11, wherein the stimulation mechanism defines a substantially rounded first end configured to be contacted by a user.

13. The skin treatment device of claim 12, further comprising a second stimulation mechanism disposed on the second side of the main body configured to transfer electrical current stimulation.

14. The skin treatment device of claim 13, wherein the second stimulation mechanism is comprised of one or more metals and defines a substantially rounded second end configured to be contacted by a user.

15. The skin treatment device of claim 14, wherein the exterior covering defines a first passageway through which the first end of the stimulation mechanism extends; and
wherein the exterior covering defines a second passageway through which the second end of the second stimulation mechanism extends.

16. The skin treatment device of claim 15, wherein the exterior covering defines a set of ridges, at least one ridge of the set of ridges being disposed adjacent the stimulation mechanism.

17. A skin treatment device having a main body configured to treat the skin of a user, comprising:
a substantially flat base configured to stand unaided on a substantially flat surface, said main body having a cross-sectional shape that is longer in a first direction substantially parallel to the base than in a second direction substantially parallel to the base;
a silicone exterior having a first side defining a first set of touch-points integrally formed with the silicone exterior, the first set of touch-points being disposed on a first side of said main body;
an oscillating motor disposed within said main body, the oscillating motor configured to produce pulsations of the skin treatment device;
a heating element disposed adjacent the first side of the main body, the heating element configured to produce heat;
a heat transfer mechanism disposed adjacent the heating element and silicone exterior, the heat transfer mechanism configured to transfer the heat to said skin;
a stimulation mechanism disposed on a second side of the silicone exterior, the stimulation mechanism configured to transfer electrical current stimulation to said skin; and
a second stimulation mechanism disposed on a second side of the silicone exterior, the second stimulation mechanism configured to transfer electrical current stimulation to said skin.

18. The skin treatment device of claim 17, wherein the main body defines an indented portion and a protrusion extending away from a surface of the indented portion;
wherein the heating element is at least partially disposed within the indented portion and adjacent the protrusion; and
wherein the heating element is comprised of one or more metals.

19. The skin treatment device of claim 18, wherein the heat transfer mechanism is at least partially disposed within the indented portion and adjacent the protrusion; and
wherein the heat transfer mechanism is disposed above the heating element within the indented portion.

20. The skin treatment device of claim 19, wherein the heat transfer mechanism is comprised of one or more metals and defines a set of nodes configured to be contacted by a user to transfer heat therapy to the user when said skin treatment device is in use.
